(19) United States
(12) Patent Application Publication
(10) Pub. No.: US 2003/0138427 A1
(43) Pub. Date: Jul. 24, 2003

(54) Modulation of plasma membrane human leukocyte elastase

(76) Inventor: Cynthia L. Bristow, New York, NY (US)

Correspondence Address: Keith D. Nowak, Esq., Lieberman & Nowak, LLP, 350 Fifth Avenue, New York, NY, 10118, US

(21) Appl. No.: 10/353,243

(22) Filed: Jan. 28, 2003

Related U.S. Application Data
(63) Continuation of application No. 09/896,743, filed on Jun. 29, 2001, now abandoned.
(60) Provisional application No. 60/216,064, filed on Jul. 5, 2000.

(51) Int. Cl. A61K 39/395; A61K 38/17; C12N 9/99

(52) U.S. Cl. 424/146.1; 514/12; 435/184

(57) ABSTRACT

A method for modulation of plasma membrane associated Human Leukocyte Elastase (HLE) to inflammatory states by interaction of HLE with an antagonist to inhibit HLE and thereby interruption in plasma associated events (e.g. HIV disease progression, bacterial infections and autoimmune diseases), which are responsive/sensitive to such inflammation. The antagonist suitable for use in this invention is designed to interact with each of the catalytic triad of the HLE plasma membranes protein and the lipid interactive amino acids of the HLE plasma membrane protein.

### RELATED APPLICATIONS

This application claims the filing date of Provisional Patent Application No. 60/216,064 filed Jul. 5, 2000.

### BACKGROUND OF THE INVENTION

1. Field of the Invention

This invention relates to a method. More specifically, this invention relates to a method for the modulation of plasma membrane associated Human Leukocyte Elastase (HLE) by interaction of said plasma membrane associated HLE with an antagonist to inhibit HLE and thereby interruption in plasma associated events (e.g. HIV disease progression, bacterial infections and autoimmune diseases), that are responsive/sensitive to inflammatory states.

2. Background of the Invention

It has been previously reported that human leukocyte elastase (HLE) is involved in plasma membrane events during stimulation of immune cells, (Bristow and Flood, T Cell Antigen Immune Complexes Demonstrating Biologic & Proteolytic Activity, Int. Immunol., Vol. 5(1):79-88 (1993). HLE is localized on the plasma membrane early in ontogeny and is granule-localized later in ontogeny, suggesting that HLE is an early differentiation marker (Borregaard and Cowland, Granules Of The Human Neutrophilic Polymorphonuclear Leukocyte, Blood, 89:3503-3521 (1997). Traditionally, HLE proteinase activity has been characterized in aqueous environments, and this has precipitated the optimization of HLE antagonists to block HLE activity in aqueous conditions. However, cell-surface lipids are known to negatively influence catalytic activity (Bangalore and Travis, Comparison Of Properties OF Membrane Bound Versus Soluble Forms Of Human Leukocyte Elastase & Cathepsin, G. Biol. Chem. Hoppe-Seyler, Vol. 375: 659-666, (1994). The inventor's own research appears to indicate/confirm that the primary function of cell-surface HLE is involved cell motility, and evidence suggests modulation of cell-surface HLE dramatically influences cellular response.

Although plasma membrane-associated HLE has been previously shown to produce membrane-associated cellular response factors, currently developed HLE antagonists have not successfully curbed these activities. For example, a cell-surface elastase has been found to mediate mitogenic signal transduction suggesting unique cellular action in response to HLE ligation. Significant historic data support a role of HLE in the genesis of atherosclerotic plaque formation. Complex interactions involving HLE result in the degradation of elastin and collagen in arterial intima during the inflammatory response have also been reported. HLE has also been shown to possess bactericidal activity.

Moreover, it has been observed by the inventor, that when cells from healthy volunteers are infected in vitro with HIV, HIV production is correlated with the cell surface density of HLE, but not to HIV receptors CD4, CXCR4, nor CCR5 (Bristow, C. L., Clin. Diagn. Lab. Immunol., Vol. 8, September 2001). HIV infection is apparently facilitated by co-patching with CD4, CXCR4, and HLE on extensions of the plasma membrane. This suggests a primary function for HLE in CD4-related events, including HIV entry and augmentation of immune response.

Notwithstanding, the foregoing observations, by both the inventors and others, of HLE involvement in various disease states, there has been little effort made to address such involvement in the context of prevention of such disease states. Accordingly, there continues to exist the need to expand the understanding of such HLE involvement in the cellular response to infectious agents, and if possible, utilize such understanding to define a prophylactic agent or mechanism to prevent or modulate such response to various disease states by control of HLE involvement in such processes.

### OBJECTS OF THE INVENTION

It is the object of the invention to remedy the above as well as related deficiencies in the prior art.

It is the principle object of this invention to provide a method for modulation of plasma membrane-associated response to infection through the use of antagonists specific for plasma membrane HLE.

It is another object of this invention to provide a method for modulation of plasma membrane-associated response to infection by interaction of HLE with an antagonist to inhibit HLE and thereby interruption in plasma associated events (e.g. response to inflammatory states, including HIV disease progression, bacterial infections and autoimmune diseases) that can result in infection or progression of a disease state.

It is yet another object of this invention to provide an HLE antagonist specific for cell surface HLE that is effective in the modulation of cell membrane-associated response to infection.

### SUMMARY OF THE INVENTION

The above and related object are achieved by providing a method for inhibition of HLE plasma membrane response to disease states by the in vivo interaction of such plasma membrane with an HLE antagonist specific for such HLE. It is understood that the phrase/term “plasma membrane” or “membrane” is used interchangeably herein and inclusive of any biologically discrete entity wherein the HLE is associated with “charged molecules” in the form of a plasma membrane or modified plasma membrane. Accordingly, the peptide antagonist of this invention is suitable for the modulation of HLE where such HLE is present in the plasma membranes of whole cells; vesicles or modified vesicles of whole cells; cellular organelles or their corresponding membranes; charged molecules associated with lipids or modified lipids; charged molecules associated with nucleic acids or modified nucleic acids; and charged molecules associated with carbohydrates or modified carbohydrates.

The peptide antagonist of this invention is specific for interaction with plasma membrane HLE so as to interrupt plasma membrane associated events characteristic of inflammatory states, (including HIV disease progression, bacterial infections and autoimmune diseases). Thus, the method and peptide antagonist of this invention is suitable in the modulation of the HLE plasma membrane response/sensitivity to HIV infection, at any stage of the illness; to modulation of endogenous and/or exogenous biological irritants which cause and/or propagate osteo and rheumatoid arthritis; atherosclerosis; diabetes; asthma; systematic lupus erythematosis; inflammatory diseases of lymphoid origin, including, but not limited to agammaglobulinemia, hypogammaglobulinemia, hypergammaglobulinemia, NK cells, T lymphocytes, B lymphocytes, thymocytes, bone marrow, or null cells; age-related illness such as dementia; anaphylactic conditions; tumors of any origin, primary or secondary origin; autoimmune diseases; infections of bacterial, viral, or other parasitic origin; demyelinating disease (e.g. MS or MLS); hemolytic anemia; inflammatory diseases of cardiovascular origins; to toxin or toxoid including, but not limited to cholera, pertussis, diphtheria, tetanus, or Escherichia coli; to a poison including, but not limited to, stings, bites, ingested poisons, or skin contact; mucosal inflammation including gastrointestinal disorders; pulmonary tissue inflammation; granulomatous disease; hepatic disorders; and, in minimizing the effects of rejection in organ transplantation including, but not limited to, human organs or xenotransplants or in transfusions or to induce immune system tolerance.

It is anticipated that the peptide antagonists of this invention shall be particularly useful in replacement therapy including, but not limited to, as replacement therapy for proteinase inhibitors.

In order to be effective in this regard, the peptide antagonist of this invention is designed to interact with each of the catalytic triad of the HLE membrane surface protein and the lipid interactive amino acids of the HLE membrane surface protein. This catalytic triad of HLE (domain 1) is composed of amino acids His (41), Asp (88), and Ser (173). Lipid-interactive amino acids of the HLE (domain 2) is composed of amino acids Phe (170), Ala(187), and Arg (191); and, these amino acids are proximal to the catalytic triad. The HLE antagonist comprise peptides which interact with membrane surface HLE, which are designed and modified to optimize their influence on these two domains of the HLE, and thereby intervene in plasma membrane-associated HLE activities.

The administration of effective amounts of HLE antagonist permit the medical management of pathologic immune responses resulting from microbial organisms, transplantation, autoimmunity, cancer, HIV infection, and many other disease states.

### DETAILED DESCRIPTION OF THE INVENTION INCLUDING PREFERRED EMBODIMENTS

The amino acids of the HLE peptide antagonists suitable for use in this invention are designated using standard three-letter abbreviations followed in parenthesis by the numerical position in the HLE protein sequence, the first amino acid being amino-terminal Ile (1). The catalytic triad of HLE (domain 1) is composed of amino acids His (41), Asp (88), and Ser (173). Lipid-interactive amino acids (domain 2) are Phe (170), Ala (187), and Arg (191), and these amino acids are proximal to the catalytic triad.

The peptides antagonists of this invention are designed and modified to optimize their influence on these two domains can be utilized to intervene in plasma membrane-associated HLE activities. As noted above, the catalytic triad of HLE is very well characterized. Peptide antagonists containing the sequence L-Ala-L-Ala-L-Pro-L-Val-chloromethylketone specifically and covalently inhibit the amidolytic activity of HLE. Hydrophobic amino acids near the catalytic triad are interactive with charged entities such as lipids and nucleic acids. Modification of such peptides in the manner described herein, thus, permits the development of more efficient antagonists interactive with lipid-associated HLE and, thereby effective interruption of plasma membrane-associated events.

The effectiveness of plasma membrane-associated HLE interactive peptide antagonists can be measured by detecting proteolytic activity, signal transduction, cellular adherence, cell motility, HIV infectivity, uropod formation, and cell surface density of HLE using isolated cell preparations. The inventor has previously identified one plasma membrane-associated HLE interactive peptide capable of diminishing in vitro HIV infectivity, (Bristow, C. L., Fiscus, S. A., Flood, P. M., and Arnold, R. R., Inhibition of HIV-1 By Modification of a Host Membrane Protease, Int.Immunol. Vol. 7:, 239-249 (1995).

Delivery of plasma membrane-associated HLE interactive peptides can involve combinatorial protein constructs, and can incorporate lipids and carbohydrates in the structural design to prolong half-life and optimize HLE-interactive efficiency. Plasma membrane-associated HLE interactive peptides can also be used as antagonists for HLE associated with plasma membranes vesicles not associated with whole cells. Moreover, plasma membrane-associated HLE interactive peptides can be used as antagonists for HLE associated with lipid moieties and charged entities not associated with plasma membranes. In addition, the plasma membrane-associated HLE interactive peptides can be used as antagonists for HLE associated with nucleic acids and particles associated with nucleic acids.

Plasma membrane-associated HLE interactive peptides will allow intervention in general inflammatory states including HIV disease progression, atherosclerosis, arthritis, asthma, organ transplantation, neoplastic diseases, bacterial infections, and autoimmune diseases.

### EXAMPLES

A peptide antagonist suitable for use in the modulation of cell surface HLE is prepared by an iterative process of mutagenesis, expression, chromatographic selection, and amplification. In this process, a gene encoding a potential binding domain, is obtained by random mutagenesis of a limited number of predetermined codons, and such gene fused to a genetic element which causes the resulting chimeric expression product to be displayed on the outer surface of a virus (e.g. a filamentous phage) or a cell. Chromatographic selection is then used to identify viruses or cells whose genome includes a fused gene coded for the protein which is bound to the chromatographic target. The foregoing technique for preparation of the peptide antagonists of this invention is more fully described in Ladner, et al. U.S. Pat. No. 5,571,698, which is herein incorporated by reference in its entirety.

The efficacy of plasma membrane-associated HLE interactive peptides prepared in the foregoing manner is confirmed in vivo using primate models. More specifically, each of two rodents that are infected with HIV are monitored for the progression of the disease, after one of them is inoculated with a peptide antagonist for HLE at levels that have been empirically determined to be effective to inhibit HIV. The progression of the HIV infection in the rodent inoculated with the peptide antagonist remains relative unchanged, and appears to regress; whereas, the rodent that is denied the peptide inoculation becomes progressively more ill and eventually dies.

The progression of the disease in the rodent receiving the HLE peptide antagonist is monitored as a function of the circulating concentration of its physiologic ligand a₁proteinase inhibitor (a₁PI, a₁antitrypsin). Ligation of cell surface HLE with a₁PI prevents detection of cell surface HLE expression. The inventor has demonstrated that cell surface HLE density and a₁PI are directly correlated with HIV produced in vitro and in vivo. Therefore, appropriately designed peptides interactive with plasma membrane-associated HLE intervene in HIV disease progression; and, the effectiveness of such intervention can be effectively monitored by determnination of the circulating concentration of its physiologic ligand a₁proteinase inhibitor (a₁PI, a₁antitrypsin).

Application of the method of this invention will, thus, allow management of pathologic immune responses resulting from microbial organisms, transplantation, autoimmunity, cancer, HIV infection, and many other disease states.

What is claimed is:

1. A method for modulation of plasma membrane-associated response to an inflammatory irritant, in the nature of a disease or pathogen or toxin, through the use of antagonists specific for plasma membrane HLE, comprising:
A. Providing a peptide antagonist specific for interaction with plasma membrane lipid-associated Human Leukocyte Elastase (HLE), said antagonist being specific for interaction with said HLE on said plasma membrane by binding to said HLE so as to inhibit plasma membrane-associated response of said host cells to an inflammatory irritant,
said peptide having at least two functional sites, one for interaction with each of a catalytic triad of the HLE cell surface protein and lipid interactive amino acids of the HLE cell surface protein, wherein said catalytic triad of HLE (domain 1) is composed of amino acids His (41), Asp (88), and Ser (173) and said lipid-interactive amino acids of the HLE (domain 2) is composed of amino acids Phe (170), Ala (187), and Arg (191), with the proviso that said lipid interactive amino acids are proximal to said catalytic triad; and
B. Contacting said plasma membrane lipid-associated Human Leukocyte Elastase (HLE), with said peptide antagonist under binding conditions so as to effect interaction of said antagonist and said plasma membrane lipid-associated Human Leukocyte Elastase (HLE), and thereby suppression of plasma membrane response to inflammation.
